(19) United States
(12) Patent Application Publication
(10) Pub. No.: US 2025/0281081 A1
(43) Pub. Date: Sep. 11, 2025

(54) WEARABLE COMPUTING DEVICE, SYSTEMS, AND METHOD FOR MEASURING SKIN AUTOFLUORESCENCE WITH AN OPTICAL SENSOR

(71) Applicant: Google LLC, Mountain View, CA (US)

(72) Inventors: Hamed Vavadi, Castro Valley, CA (US); Kaiyuan Yao, Fremont, CA (US); Herschel Max Watkins, Sausalito, CA (US); Lindsey Michelle Sunden, San Francisco, CA (US); Peter Winthrop Richards, San Francisco, CA (US); Shelten Gee Jao Yuen, Berkeley, CA (US)

(21) Appl. No.: 18/599,936

(22) Filed: Mar. 8, 2024

(51) Int. Cl. A61B 5/1455; A61B 5/0205; A61B 5/024; A61B 5/145; G16H 40/67

(52) U.S. Cl. CPC A61B 5/1455; A61B 5/0205; G16H 40/67; A61B 5/02416; A61B 5/02438; A61B 5/14546; A61B 2562/0238; A61B 2562/066; A61B 2562/185

(57) ABSTRACT

A wearable computing device for measuring skin autofluorescence is provided. The device includes a skin autofluorescence sensor having one or more emitters configured to output one or more emitted light signals, a first detector configured to receive a first returned light signal, the first detector including an optical long pass filter, and a second detector configured to receive a second returned light signal. In addition, a light blocking material is disposed between the one or more emitters and the first detector, the second detector, or both. The device also includes a processor configured to calculate a skin autofluorescence level based on a measured intensity level of the first returned light signal and a measured intensity level of the second returned light signal. A method of measuring skin autofluorescence using the device is also provided.

### FIELD

The disclosure relates generally to wearable computing devices. More particularly, the disclosure relates to wearable computing devices that utilize optical sensors to measure skin autofluorescence (SAF) via a diffuse optical method.

### BACKGROUND

Skin autofluorescence can be used to non-invasively measure levels of advanced glycation end products (AGEs) that are present below a surface of a person's skin. AGEs are biomarkers that have been associated with aging and cardiovascular health and have also been implicated in such conditions as diabetes, atherosclerosis, kidney disease, and Alzheimer's disease. In particular, skin autofluorescence can be strongly correlated with health outcomes and could be used in conjunction with other physiologic data (e.g., heart rate and oxygen saturation (SpO₂)) to provide information to a person about the person's health. However, the accuracy of SAF measurements can be impacted by skin temperature, skin pressure, the non-homogeneous nature of AGE distribution in the skin, the level of melanin in the skin, and other factors. Moreover, existing advanced glycation end (AGE) readers are large and costly so that their use is limited to occasional spot checks in the clinical setting, leading to inability to accurately track trends in SAF levels. Further, existing AGE readers are based on a reflection geometry where a majority of the returned signals are from a surface of skin. In other words, the existing readers use an optical scheme in which the area of skin that the light source is shined onto and the detection area are the same. Thus, the majority of the autofluorescence portion of the returned light signal is coming from the surface of the skin. Such an arrangement creates surface artifacts that negatively impact the accuracy of traditional SAF readings.

As such, a need exists for a device and method of measuring SAF continuously and in an accurate manner in which the light penetrates through the skin rather than being mostly reflected off the skin's surface in order to provide a more accurate quantification of SAF levels based on diffuse fluorescence, which can then be used to provide information about a user's health.

### SUMMARY

Aspects and advantages of embodiments of the disclosure will be set forth in part in the following description, or can be learned from the description, or can be learned through practice of the example embodiments.

In one aspect, a wearable computing device for measuring skin autofluorescence is provided. The wearable computing device includes a skin autofluorescence sensor that itself includes one or more emitters configured to output one or more emitted light signals; a first detector configured to receive a first returned light signal, the first detector including an optical long pass filter; and a second detector configured to receive a second returned light signal. The wearable computing device also includes a light blocking material disposed between the one or more emitters and the first detector, the second detector, or both; and a processor configured to calculate a skin autofluorescence level based on a measured intensity level of the first returned light signal and a measured intensity level of the second returned light signal.

In some implementations, the one or more emitted light signals can have a wavelength ranging from about 300 nanometers to about 900 nanometers.

In some implementations, the first detector can be separated from the one or more emitters by a distance ranging from about 0.5 millimeters to about 6 millimeters.

In some implementations, the second detector can be separated from the one or more emitters by a distance ranging from about 0.5 millimeters to about 6 millimeters.

In some implementations, the skin autofluorescence level can be measured continuously.

In some implementations, the skin autofluorescence sensor can be in direct contact with the user's skin.

In some implementations, the one or more emitted light signals can penetrate to the user's dermis.

In some implementations, the one or more emitted light signals can penetrate to the user's subcutaneous tissue.

In some implementations, the one or more emitted light signals can penetrate beneath the user's skin by an average distance of about 0.01 millimeters to about 3 millimeters.

In some implementations, the wavelength of the one or more emitted light signals can range from about 350 nanometers to about 500 nanometers. Further, the optical long pass filter can prevent light having a wavelength of, for example, less than about 500 nanometers from reaching the first detector. In other words, regardless of the wavelength of the emitted light signal, the optical long pass filter can prevent light having a wavelength that is equal to the wavelength of the one or more emitted light signals emitted by the one or more emitters from reaching the first detector.

In some implementations, the wearable computing device can also include a photoplethysmography (PPG) sensor.

In another aspect, a method for measuring skin autofluorescence with a wearable computing device is provided. The method includes emitting, by one or more emitters of a skin autofluorescence sensor of the wearable computing device, one or more emitted light signals; obtaining, by a first detector of the skin autofluorescence sensor of the wearable computing device, a first returned light signal, the first detector including an optical long pass filter; obtaining, by a second detector of the skin autofluorescence sensor of the wearable computing device, a second returned light signal; calculating, by a processor, a skin autofluorescence level based on a measured intensity level of the first returned light signal and a measured intensity level of the second returned light signal.

In some implementations, the one or more emitted light signals can have a wavelength ranging from about 300 nanometers to about 900 nanometers.

In some implementations, the first detector can be separated from the one or more emitters by a distance ranging from about 0.5 millimeters to about 6 millimeters.

In some implementations, the second detector can be separated from the one or more emitters by a distance ranging from about 0.5 millimeters to about 6 millimeters.

In some implementations, the skin autofluorescence level can be measured continuously.

In some implementations, the skin autofluorescence sensor can be in direct contact with the user's skin.

In some implementations, the one or more emitted light signals can penetrate to the user's dermis, the user's subcutaneous tissue, or both.

In some implementations, the one or more emitted light signals can penetrate beneath the user's skin by an average distance of about 0.01 millimeters to about 3 millimeters.

In some implementations, the wavelength can range from about 350 nanometers to about 500 nanometers.

In some implementations, the optical long pass filter can prevent light having a wavelength of, for example, less than about 500 nanometers from reaching the first detector. In other words, regardless of the wavelength of the emitted light signal, the optical long pass filter can prevent light having a wavelength that is equal to the wavelength of the one or more emitted light signals emitted by the one or more emitters from reaching the first detector.

In some implementations, the wearable computing device by which the method is carried out can further include a photoplethysmography (PPG) sensor.

These and other features, aspects, and advantages of various embodiments of the disclosure will become better understood with reference to the following description, drawings, and appended claims. The accompanying drawings, which are incorporated in and constitute a part of this specification, illustrate example embodiments of the disclosure and, together with the description, serve to explain the related principles.

### BRIEF DESCRIPTION OF THE DRAWINGS

Detailed discussion of example embodiments directed to one of ordinary skill in the art is set forth in the specification, which makes reference to the appended drawings, in which:

FIG. 1 depicts a perspective front view of an example wearable computing device according to some implementations of the present disclosure.

FIG. 2 depicts a rear view of an example wearable computing device according to some implementations of the present disclosure.

FIG. 3 depicts an example block diagram of the wearable computing device according to some implementations of the present disclosure.

FIG. 4 is a cross-sectional schematic illustration of a portion of the autofluorescence sensor of the wearable computing device according to one embodiment of the disclosure when the sensor is placed in direct contact with a user's skin, particularly showing the resulting optical path (e.g., emitted light signal and returned light signal) used to measure skin autofluorescence.

FIG. 5 is a graph illustrating the depth in the tissue to which light has penetrated when it is emitted from the emitter and reflected to the detector of the wearable computing device of FIG. 4.

FIG. 6 is a graph illustrating the skin autofluorescence signal to reference signal ratio for various skin tones for the diffusive optical geometry used in the wearable computing device of FIG. 4.

FIG. 7 is a cross-sectional schematic illustration of a portion of an autofluorescence sensor used in prior art devices where the sensor is not placed in direct contact with a user's skin, particularly showing the resulting optical path (e.g., emitted light signal and returned light signal) used to measure skin autofluorescence.

FIG. 8 is a graph illustrating the depth in the tissue to which light has penetrated when it is emitted from the emitter and reflected to the detector of the prior art device of FIG. 7.

FIG. 9 is a graph illustrating the skin autofluorescence signal to reference signal ratio for various skin tones for the prior art reflective optical geometry used in the prior art device of FIG. 7.

FIG. 10 is a graph comparing the advanced glycation end product (AGE) reader score of the prior art on the x-axis (which is the fluorescence signal divided by the reflectance signal) to the measured fluorescence using the diffuse reflectance arrangement of the present disclosure on the y-axis.

FIG. 11 is a bar graph showing the variability and lack of accuracy of advanced glycation end product (AGE) reader score with prior art devices that do not provide for continuous measurement.

FIG. 12 illustrates a flow diagram for a method for measuring skin autofluorescence using a wearable computing device.

Reference numerals that are repeated across plural figures are intended to identify the same features in various implementations.

### DETAILED DESCRIPTION

Reference now will be made to embodiments of the disclosure, one or more examples of which are illustrated in the drawings. Each example is provided by way of explanation of the disclosure and is not intended to limit the disclosure. In fact, it will be apparent to those skilled in the art that various modifications and variations can be made to disclosure without departing from the scope or spirit of the disclosure. For instance, features illustrated or described as part of one embodiment can be used with another embodiment to yield a still further embodiment. Thus, it is intended that the disclosure covers such modifications and variations as come within the scope of the appended claims and their equivalents.

Terms used herein are used to describe the example embodiments and are not intended to limit and/or restrict the disclosure. The singular forms “a,” “an” and “the” are intended to include the plural forms as well, unless the context clearly indicates otherwise. In this disclosure, terms such as “including”, “having”, “comprising”, and the like are used to specify features, numbers, steps, operations, elements, components, or combinations thereof, but do not preclude the presence or addition of one or more of the features, elements, steps, operations, components, or combinations thereof.

It will be understood that, although the terms first, second, third, etc., may be used herein to describe various elements, the elements are not limited by these terms. Instead, these terms are used to distinguish one element from another element. For example, without departing from the scope of the disclosure, a first element may be termed as a second element, and a second element may be termed as a first element.

The term “and/or” includes a combination of a plurality of related listed items or any item of the plurality of related listed items. For example, the scope of the expression or phrase “A and/or B” includes the item “A”, the item “B”, and the combination of items “A and B”.

In addition, the scope of the expression or phrase “at least one of A or B” is intended to include all of the following: (1) at least one of A, (2) at least one of B, and (3) at least one of A and at least one of B. Likewise, the scope of the expression or phrase “at least one of A, B, or C” is intended to include all of the following: (1) at least one of A, (2) at least one of B, (3) at least one of C, (4) at least one of A and at least one of B, (5) at least one of A and at least one of C, (6) at least one of B and at least one of C, and (7) at least one of A, at least one of B, and at least one of C.

Generally speaking, the present disclosure is directed to a wearable computing device that includes an optical sensor for measuring skin autofluorescence. The optical sensor includes one or more emitters (e.g., a light emitter such as a light emitting diode (LED)) configured to output one or more emitted light signals having a wavelength ranging from about 300 nanometers to about 900 nanometers, a first detector configured to receive a first returned light signal, the first detector including an optical long pass filter, and a second detector configured to receive a second returned light signal. In addition, a light blocking material is disposed between the one or more emitters and the first detector, the second detector, or both. The device also includes a processor configured to determine a ratio of a measured intensity level of the first returned light signal to a measured intensity level of the second returned light signal to determine a level of autofluorescence present below a surface of a user's skin. A method of measuring skin autofluorescence using the device is also provided.

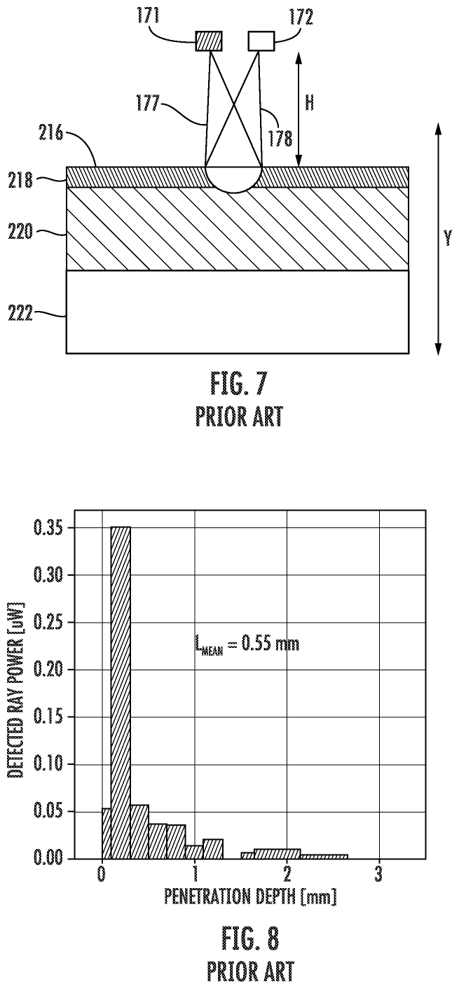

Without intending to be limited by any particular theory, the present inventors have found that the optical geometry of the wearable computing devices contemplated by the present disclosure are particularly amenable to measuring skin autofluorescence. In particular, the devices contemplated by the present disclosure produce more accurate readings compared to existing, stand-alone advanced glycation end product (AGE) readers used for measuring skin autofluorescence. This is because existing AGE readers utilize reflective optical geometry and utilize a light emitter and a light detector that are in close proximity to each other laterally (in the x-direction) and that are spaced several centimeters away from the user's skin vertically (in the y-direction), where it has been estimated that 85% of the optical power detected by the light detector is reflected from above the skin's surface. Such specularly-reflected photons experience minimal interaction with tissue and contribute to a large background signal (see FIG. 7).

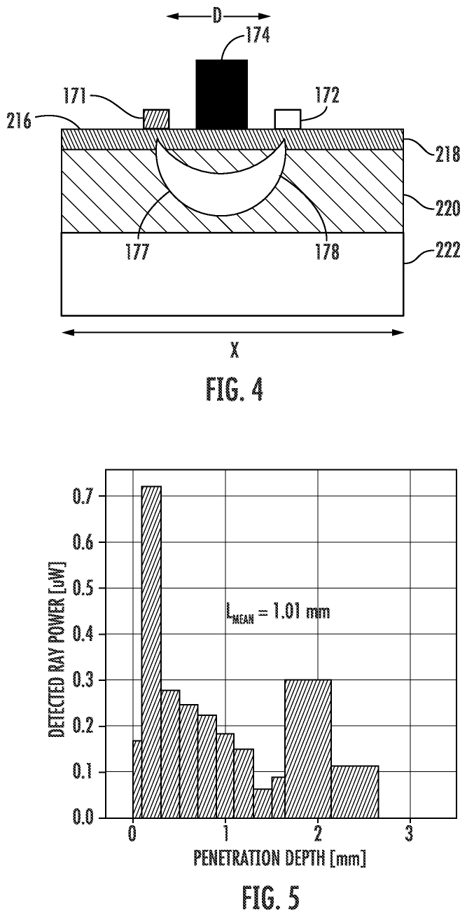

Meanwhile, the devices contemplated by the present disclosure utilize a diffuse optical geometry where the light emitter and the light detector are separated from each other laterally by a sufficient distance and are also placed in close proximity, or in contact with, the user's skin (see FIG. 4). Moreover, light blocking structures can also be disposed between the light emitter and light detector to minimize specular reflection and the reflection of light directly off the skin's surface and into the light detector. As such, light is forced to dive into the user's tissue with a banana-shaped path trajectory and at a deep penetration depth. Since a majority of skin autofluorescence signal originates from collagen cross-linking, and collagen is most abundant in the subsurface dermis layer beneath the epidermis located at the surface of the user's skin, it follows that the diffuse geometry contemplated by the devices of the present disclosure provide a strong skin autofluorescence signal compared to the existing reflective geometry that is known in the prior art. In addition, the devices and methods of the present disclosure contemplate that the skin autofluorescence measurements can be taken continuously while the user is wearing the wearable computing device, which can improve accuracy of skin autofluorescence measurements compared to existing AGE readers. For instance, the devices and methods of the present disclosure contemplate taking measurements continuously while the device is being worn, which includes taking intermittent measurements throughout the day that can be spaced apart by a time frame of about 0.0001 seconds to about 24 hours, or any range therebetween, as opposed to taking one discrete measurements as is done with existing AGE readers.

Further, the present disclosure contemplates a skin autofluorescence sensor arrangement that utilizes at least one light emitter and at least two light detectors, where one light detector includes a long pass filter that blocks the non-fluorescent portion of the returned light signal in order to only focus on measuring the intensity of the fluorescent portion of the returned light signal, which further improves the accuracy of the skin autofluorescence readings. For instance, the optical long pass filter prevents light having a wavelength of less than about 450 nanometers from reaching the detector that includes the long pass filter.

Example aspects of the present disclosure are directed to a wearable computing device that can be worn, for example, on a user's wrist. The wearable computing device includes an optical sensor that includes a skin autofluorescence sensor that is configured to generate a returned light signal that is indicative of a biometric (e.g., skin autofluorescence level) of the user. The autofluorescence sensor includes one or more light emitters that can include one or more light sources (e.g., light emitting diodes (LEDs)) configured to emit a light signal toward a body part of the user when the wearable computing device is worn by the user. The skin autofluorescence sensor further includes two or more detectors (e.g., photodiodes) configured to receive a reflection of the light emitted toward the body part. The ratio of these two returned light signals is used to determine a skin autofluorescence level of the user, which can then be used to determine various health metrics associated with, but not limited to, cardiovascular health, diabetes, atherosclerosis, kidney disease, and Alzheimer's disease.

The optical sensors of the wearable computing device can also include a PPG sensor that is configured to generate a PPG signal indicative of a biometric (e.g., heart rate) of the user. The PPG sensor includes one or more light emitters that can include one or more light sources (e.g., light emitting diodes (LEDs)) configured to emit light toward a body part of the user when the wearable computing device is worn by the user. The PPG sensor further includes one or more detectors (e.g., photodiodes) configured to receive a reflection of the light emitted toward the body part. It should be understood that the PPG signal is the reflection of the light.

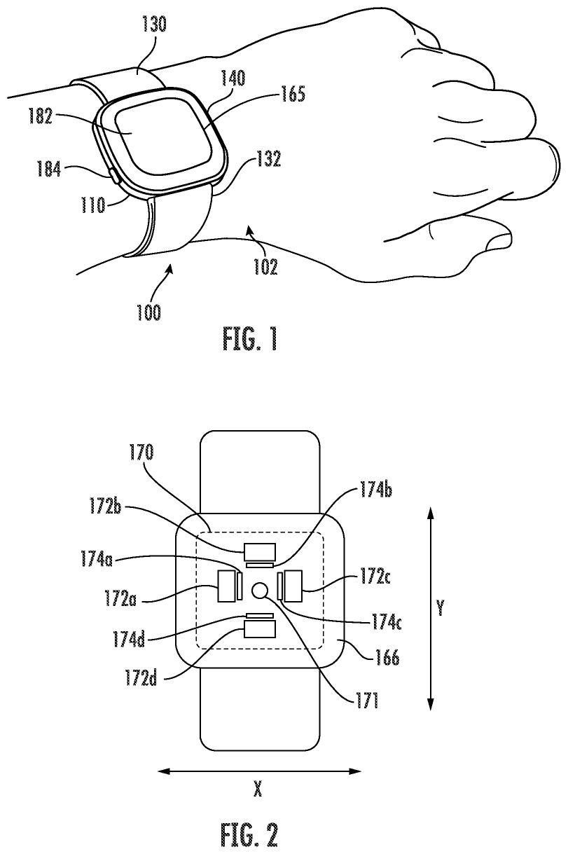

Referring now to the drawings, FIGS. 1 through 4 illustrate examples of a wearable computing device 100 according to various examples of the disclosure. The wearable computing device 100 can be worn, for example, on a body part 102 (e.g., an arm, wrist, etc.) of a user. The wearable computing device 100 includes a body 110 having an outer facing surface 165, which can be referred to as the front of the wearable computing device 100, and a skin contacting surface 166, which can be referred to as the back of the wearable computing device 100. Furthermore, the body 110 defines a cavity (not shown) between the outer facing surface 165 and the skin contacting surface 166 in which one or more electronic components (e.g., disposed on one or more printed circuit boards) are disposed. The wearable computing device 100 includes a printed circuit board (not shown) disposed within the cavity. Furthermore, one or more electronic components are disposed on the printed circuit board. The wearable computing device 100 can further include a battery that is disposed within the cavity defined by the body 110.

In FIG. 1 the wearable computing device 100 includes a first band 130 and a second band 132. As shown, the first band 130 is coupled to the body 110 at a first location thereon. Conversely, the second band 132 is coupled to the body 110 at a second location thereon. Furthermore, the first band 130 and the second band 132 can be coupled to one another to secure the body 110 to the body part 102 of the user.

In some examples, the first band 130 can include a buckle or clasp (not shown). Additionally, the second band 132 can include a plurality of apertures (not shown) spaced apart from one another along a length of the second band 132. In such implementations, a prong of the buckle associated with the first band 130 can extend through one of the plurality of openings defined by the second band 132 to couple the first band 130 to the second band 132. It should be appreciated that the first band 130 can be coupled to the second band 132 using any suitable type of fastener. For example, in an embodiment, the first band 130 and the second band 132 can include a magnet. In such implementations, the first band 130 and the second band 132 can be magnetically coupled to one another to secure the body 110 to a body part 102 (e.g., an arm) of the user.

In FIG. 1, the wearable computing device 100 includes a cover 140 positioned on the body 110 so that the cover 140 is positioned on top of a display 182. In this manner, the cover 140 can protect the display 182 from being scratched. In an embodiment, the wearable computing device 100 can include a seal (not shown) positioned between the body 110 and the cover 140. For instance, a first surface of the seal can contact the body 110 and a second surface of the seal can contact the cover 140. In this manner, the seal between the body 110 and the cover 140 can prevent a liquid (e.g., water) from entering the cavity defined by the body 110.

It should be understood that the cover 140 can be optically transparent so that the user can view information being displayed on the display 182. For instance, in an embodiment, the cover 140 can include a glass material. It should be understood, however, that the cover 140 can include any suitable optically transparent material.

Referring to FIG. 2, the wearable computing device 100 further includes various sensors 170 (e.g., optical sensors) that are disposed within the cavity defined by the body 110 or on a surface of the body 110. For example, the sensors 170 may include one or more skin autofluorescence sensors and/or one or more photoplethysmography (PPG) sensors disposed on a skin contacting surface 166 of the body 110. The skin autofluorescence sensors can, for example, be used to monitor for advanced glycation end products below a surface of the user's skin. The skin autofluorescence sensors can include one or more light sources or emitters 171 (e.g., light-emitting diodes (LEDs)) and one or more light detectors 172a-d (e.g., photodiodes). Meanwhile, the PPG sensor(s) can, for example, be used to monitor a heart rate of the user. The PPG sensor(s) can also include one or more light sources or emitters 171 (e.g., light-emitting diodes (LEDs)) and one or more light detectors (e.g., photodiodes) 172a-d. In FIG. 2, a skin contacting surface 166 (e.g., a rear surface) of an example wearable computing device 100 is illustrated according to one or more example embodiments of the disclosure. It should be understood that the although only one light source or emitter 171 is shown, multiple emitters 171 can be utilized, where one light source or emitter 171 can be associated with the skin autofluorescence sensor portion of the optical sensor 170 and another light source or emitter (not shown) can be associated with the PPG sensor portion of the optical sensor 170. In particular, the one or more light sources or emitters 171 can be any suitable light emitting diode and can emit a light signal having a wavelength ranging from about 300 nanometers to about 900 nanometers, such as from about 325 nanometers to about 700 nanometers, such as from about 350 nanometers to about 500 nanometers. In one particular embodiment, the one or more light sources or emitters 171 for the skin autofluorescence sensor portion of the optical sensor 170 can be a near-ultraviolet LED, meaning it emits light having a wavelength ranging from about 350 nanometers to about 450 nanometers.

Meanwhile, referring to FIGS. 2 and 4, the two or more light detectors 172 for the skin autofluorescence sensor portion of the optical sensor 170 can be any combination of detectors 172a, 172b, 172c, and/or 172d, so long as a light blocking material 174a, 174b, 174c, and/or 174d is disposed between the light source or emitter 171 and any combination of the detectors 172a, 172b, 172c, and/or 172d that are utilized for the skin autofluorescence sensor portion of the optical sensor 170. The light blocking material 174a, 174b, 174c, and/or 174d can be made of any suitable material that prevents the light emitted from the emitter 171 and reflected off the surface of the user's skin 216 from reaching any of the detectors 172a, 172b, 172c, and/or 172d, which could affect the accuracy of the skin autofluorescence measurements by the optical sensor 170. For instance, the light blocking material 174a, 174b, 174c, and/or 174d can be an opaque material, such as an opaque plastic or composite material. Further, the emitter 171 and any of the detectors 172a, 172b, 172c, and/or 172d utilized in the skin autofluorescence portion of the optical sensor 170 are spaced apart from each other by a distance D ranging from about 0.5 millimeters to about 6 millimeters, such as from about 0.75 millimeters to about 5 millimeters, such as from about 1 millimeter to about 4 millimeters in the X (horizontal) direction or Y (vertical) direction, where the distance is measured from a edge of the emitter 171 to an edge of any one of the detectors 172, as shown in FIG. 4. Furthermore, more than one light source or emitter 171 (e.g., a LED) may be included such that different detectors may be combined with different LEDs and/or each detector may be combined with one or more LEDs to output a respective skin autofluorescence.

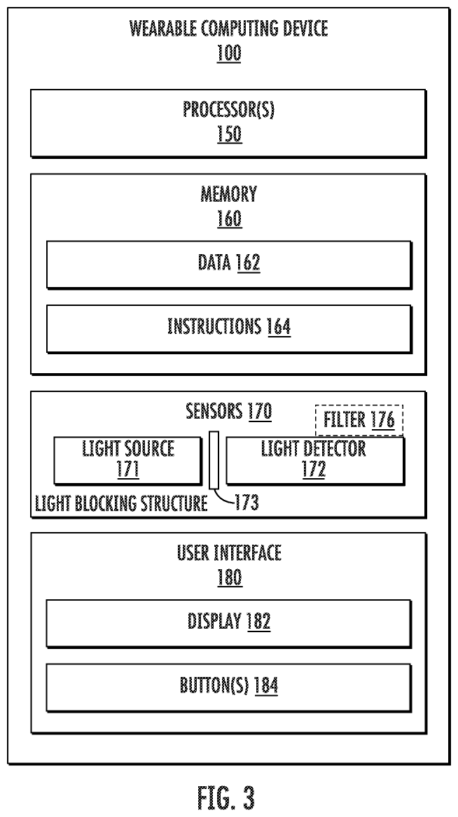

In addition, assuming, for example, that the autofluorescence portion of the optical sensor 170 utilizes emitter 171 and detectors 172a and 172b, one of the detectors 172a includes an optical long pass filter 176 (see FIG. 3). The optical long pass filter 176 can, in some embodiments, prevent ultraviolet light (e.g., a light signal having a wavelength of less than about 500 nanometers, such as less than about 450 nanometers, such as less than about 425 nanometers) from reaching detector 172a, although it is to be understood that the optical long pass filter 176 can, in other embodiments, alternatively be used to block light having a wavelength that is equal to the wavelength of the emitted light signal 177, regardless of what that wavelength is, from reaching detector 172a. In some embodiments, the long pass filter can be formed by coating a filtering material onto a silicon photodiode, where such coating materials can include silicon dioxide, zinc oxide, polycarbonate, and combinations thereof. In this manner, in the embodiment where the emitter 171 is an ultraviolet light emitting diode, detector 172a may only detect light having a wavelength of greater than 425 nanometers, such as greater than about 450 nanometers, such as greater than about 500 nanometers, where such light is associated with the fluorescent portion of the light signal's wavelength spectrum that is of interest in measuring skin autofluorescence. In other words, the optical long pass filter can prevent light having a wavelength that is equal to the wavelength of the one or more emitted light signals 177 emitted by the one or more emitters 171 from reaching detector 172a.

In particular and referring to FIG. 4, when an emitted light signal 177 is emitted from emitter 171 that can be in direct contact with the user's surface of skin 216 or spaced apart from the user's surface of skin by a distance of from about 0 millimeters to about 0.5 millimeters in the event that there is a small gap due to loss of contact during movement of the wearable computing device, the user's skin absorbs most of the emitted light signal 177 and reflects some of the emitted light signal 177, while some of the emitted light signal 177 is shifted to a longer wavelength by advanced glycation end products (AGEs) below the epidermis 218, resulting in a returned light signal 178 that has, inter alia, a fluorescent component. The average distance by which the emitted light signal 177 can penetrate beneath the user's surface of skin 216 can range from about 0.01 millimeters to about 3 millimeters, such as from about 0.05 millimeters to about 2 millimeters, such as from about 0.1 millimeters to about 2.5 millimeters. An intensity level of all components of the returned light signal 178 is what would normally be measured or determined by the detector 172a, but to obtain an accurate reading that focuses on the fluorescent component only, which is correlated to the level of AGEs present below the skin at the dermis 220 and/or subcutaneous tissue 222 layers of the skin, the long pass filter 176 is utilized to block out non-fluoresced light so that only an intensity level of the fluoresced portions of the returned light signal 178 is determined. Meanwhile the other detector 172b receives the full spectrum of wavelengths from its returned light signal since no filter is utilized with the detector 172 and determines an intensity level of the returned light signal 178. From the intensity level measurements obtained by the detectors 172a and 172b, a ratio of what portion of the light signal's intensity is associated with the fluorescent component, and hence AGEs, can be calculated via one or more processors associated with the wearable computing device 100 to determine a level of skin autofluorescence present below a surface of the user's skin. This level can then be correlated to a level of AGEs present, which can be used to determine various health risks or conditions as described above.

In addition to the optical sensor 170 including a skin autofluorescence portion, the optical sensor 170 can also include a PPG portion that can include one or more PPG sensors. Each PPG sensor may correspond to a combination of one or more light sources or emitters 171 and one or more detectors 172a, 172b, 172c, and/or 172d. For example, the wearable computing device may include two or more PPG sensors. Furthermore, more than one light source or emitter 171 (e.g., a LED) may be included such that different detectors 172a, 172b, 172c, and/or 172d may be combined with different LEDs and/or each detector may be combined with one or more LEDs to output a respective PPG signal.

It should be understood that the various light sources or emitters 171 and/or the various light detectors 172a, 172b, 172c, and/or 172d may be used in obtaining skin autofluorescence readings, PPG readings, or both and that the skin autofluorescence and/or PPG readings can be measured or determined continuously. Moreover, it should be understood that although only one light source or emitter 171 is shown, multiple light sources or emitters 171 are contemplated by the present disclosure. In addition, the arrangement of the various light sources or emitters 171 and detectors 172, 172a, 172b, 172c, and/or 172d is not limited to the arrangement shown in FIG. 2. For example, in FIG. 2, the plurality of detectors may be spaced apart from each other at regular or irregular intervals. In FIG. 2, light source (e.g. LED) 171 is disposed in a central portion of the optical sensor 170. Detectors 172a and 172c are spaced apart from light source or emitter 171 and light detectors 172b and 172d along the X-direction, and detectors 172b and 172 are spaced about from light source or emitter 171 and detectors 172a and 172c along the Y-direction. However, the configuration of the detectors and light source may be different from that illustrated in FIG. 2, and the disclosure is not limited to the example of FIG. 2.

FIG. 3 illustrates an example block diagram of the wearable computing device 100 according to one or more example embodiments of the disclosure. In FIG. 3, the wearable computing device 100 includes one or more processors 150, one or more memory devices 160, one or more sensors 170 (e.g., optical sensors having a skin autofluorescence portion and a PPG portion) that include one or more light sources or emitters 171, one or more light detectors 172, one or more light blocking structures 173, one or more filters 176, (e.g., the components of the skin autofluorescence sensors and PPG sensors discussed above), and a user interface 180.

For example, the one or more processors 150 can be any suitable processing device that can be included in a wearable computing device 100. For example, such a processor 150 may include one or more of a processor, processor cores, a controller and an arithmetic logic unit, a central processing unit (CPU), a graphics processing unit (GPU), a digital signal processor (DSP), an image processor, a microcomputer, a field programmable array, a programmable logic unit, an application-specific integrated circuit (ASIC), a microprocessor, a microcontroller, etc., and combinations thereof, including any other device capable of responding to and executing instructions in a defined manner. The one or more processors 150 can be a single processor or a plurality of processors that are operatively connected, for example in parallel.

The one or more memory devices 160 can include one or more non-transitory computer-readable storage mediums, such as such as a Read Only Memory (ROM), Programmable Read Only Memory (PROM), Erasable Programmable Read Only Memory (EPROM), and flash memory, a USB drive, a volatile memory device such as a Random Access Memory (RAM), a hard disk, floppy disks, a blue-ray disk, or optical media such as CD ROM discs and DVDs, and combinations thereof. However, examples of the one or more memory devices 160 are not limited to the above description, and the one or more memory devices 160 may be realized by other various devices and structures as would be understood by those skilled in the art.

The one or more memory devices 160 can include data 162 and instructions 164 that can be retrieved, manipulated, created, or stored by the one or more processor(s) 150.

In FIG. 3, the wearable computing device 100 includes a user interface 180 configured to receive an input from a user (e.g., via a touch input such as a thumb, finger, or an input device such as a stylus or pen) or from any of the sensors 170 (e.g., data associated with the intensity levels of the returned light signals). The wearable computing device 100 may execute a function in response to receiving the input from the user (e.g., checking health information about the user such as a blood pressure, making and/or receiving a phone call, sending and/or receiving a text message, obtaining a current time, setting a timer, a stopwatch function, controlling an external device such as a home appliance, and the like) or from any of the sensors 170 (e.g., data associated with the intensity levels of the returned light signals).

In FIG. 3, the user interface 180 includes the display 182 which displays information viewable by the user (e.g., time, date, biometric information, notifications, etc.). For example, the display 182 may be a non-touch sensitive display or a touch-sensitive display. The display 182 may include a liquid crystal display (LCD), a light emitting diode (LED) display, an organic light emitting diode (OLED) display, active matrix organic light emitting diode (AMOLED), flexible display, 3D display, a plasma display panel (PDP), a cathode ray tube (CRT) display, and the like, for example. However, the disclosure is not limited to these example displays and may include other types of displays. The display 182 may have a square or rectangular shape, or may be annular in shape (e.g., elliptical, circular, etc.). However, the shape of the display 182 is not limited thereto.

The user interface 180 may additionally, or alternatively, include one or more buttons 184 to receive an input from a user by the user applying a force to the button 184. The button 184 may be included on one or more peripheral sides of the wearable computing device 100 as shown in FIG. 1, for example. The button 184 may include mechanical components and/or electrical circuitry to implement a function of the wearable computing device 100 (e.g., setting a time, changing a setting and/or view of the display 182, selecting an option displayed on the display 182).

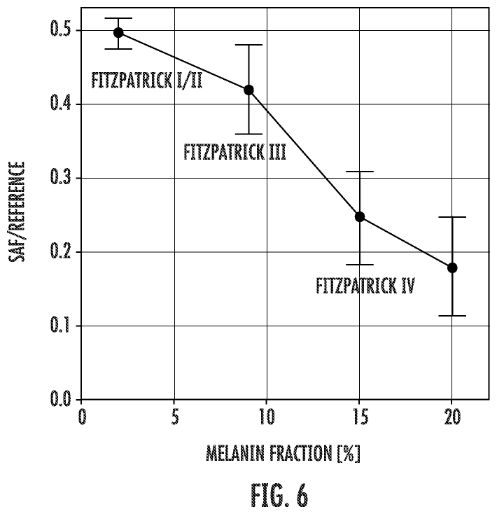

Referring now to FIGS. 4-9, the differences in the penetration depth and accuracy of the wearable devices of the present disclosure compared to existing skin autofluorescence devices is shown. In particular, FIG. 4 is a cross-sectional schematic illustration of a portion of the autofluorescence portion of the optical sensor of the wearable computing device according to one embodiment of the disclosure when the sensor is placed in direct contact with a surface of a user's skin 216, particularly showing the resulting optical path (e.g., emitted light signal 177 and returned light signal 178) from a light source or emitter 171 to a light detector 172 used to measure skin autofluorescence, where a light blocking structure 174 prevents the emitted light signal 177 from overlapping with the detected light signal 178. As shown, the optical path is able to penetrate below the epidermis 218 at the skin's surface 216 to the dermis 220. Further, although not shown, it is contemplated that at least a portion of the optical path can also reach the subcutaneous tissue 222. FIG. 5 is a graph illustrating the depth in the tissue to which light has penetrated when it is emitted from the emitter and reflected to the detector of the wearable computing device of FIG. 4. FIG. 6 is a graph illustrating the skin autofluorescence signal to reference signal ratio for various skin tones for the diffusive optical geometry used in the wearable computing device of FIG. 4.

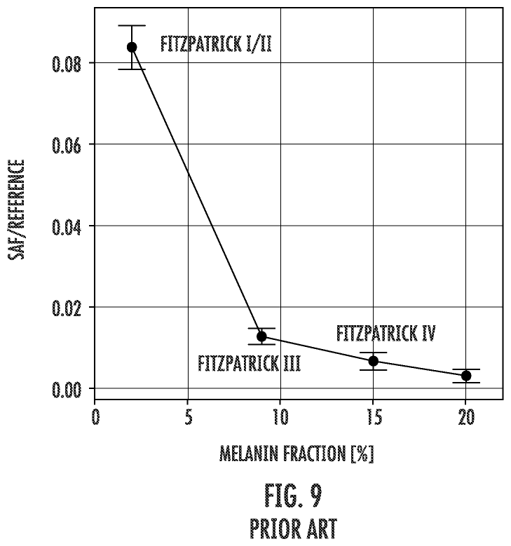

Meanwhile, FIG. 7 is a cross-sectional schematic illustration of a portion of an autofluorescence sensor used in prior art devices where the sensor is not placed in direct contact with a surface of a user's skin 216 and is instead separated by a height H, particularly showing the resulting optical path (e.g., emitted light signal 177 and returned light signal 178) used to measure skin autofluorescence. As shown, the optical path is not able to penetrate below the epidermis 218 at the skin's surface 216 to the dermis 220, and there is a significant amount of overlap between the emitted light signal 177 and the returned light signal 178, which can be attributed to the reflection off the skin's surface 216 as well as the absence of any light blocking structures 174 such as those shown in FIG. 4. FIG. 8 is a graph illustrating the depth in the tissue to which light has penetrated when it is emitted from the emitter and reflected to the detector of the prior art device of FIG. 7. FIG. 9 is a graph illustrating the skin autofluorescence signal to reference signal ratio for various skin tones for the prior art reflective optical geometry used in the prior art device of FIG. 7.

In summary, the comparison between the graphs in FIGS. 5-6 and 8-9 illustrates that using wearable devices of the present disclosure that are in direct contact with a user's skin to measure skin autofluorescence exhibits great improvement to existing SAF devices, due, in part, to the optical geometry of the wearable devices of the present disclosure. Existing medical AGE readers like the one shown in FIG. 7 use a reflective geometry, where the light source or emitter 171 and the light detector 172 are placed close together and the biosensor board is elevated several centimeters away from the users' skin surface 216 by a height H. Simulation results show that 85% of the optical power detected by the light detector 172 is reflected from above the skin's surface 216. These specularly-reflected photons experience minimal interaction with tissue and contribute to a large background signal. In contrast, the wearable device of the present disclosure and illustrated in FIG. 4 employs a diffusive optical geometry where the light source or emitter 171 and the light detector 172 are laterally separated by a distance D that can range up to about several millimeters in the X-direction and are placed in close proximity to users' skin 216. Light blocking structures 174 are also implemented to minimize specular reflection. Thus, the light emitted from the light source is forced to dive into the tissue beneath the skin with a banana-shaped trajectory and deep penetration depth, as exhibited in FIG. 5, which shows a much greater penetration depth than FIG. 8, which refers to the AGE readers of the prior art.

Since a majority of SAF signal originates from collagen crosslinking, and collagen is most abundant in the subsurface dermis layer 220 rather than the epidermis 218, the diffusive geometry described herein provides for a stronger SAF signal compared to a reflective geometry. This is numerically verified by a Monte Carlo simulation of SAF. FIGS. 5 and 8 compare the penetration depth, and FIGS. 6 and 9 compare the simulated ratio of SAF signal intensity over ultraviolet reference signal intensity for the two geometries. The diffusive design of the present disclosure (FIG. 4) shows an increased penetration depth in FIG. 5 compared to the penetration depth of the reflective design of the prior art (FIG. 7) shown in FIG. 8. Further, the diffusive design of the present disclosure exhibits an approximately 5-10 fold increase of SAF signal to reference signal ratio across all Fitzpatrick skin tones with higher advantage seen for darker skin tone users compared to existing AGE readers shown in FIG. 5, as illustrated in a comparison of FIGS. 6 and 9.

Further, FIG. 10 is a graph comparing the advanced glycation end product (AGE) reader score of the prior art on the x-axis (which is the fluorescence signal divided by the reflectance signal) to the measured fluorescence using the diffuse reflectance arrangement of the present disclosure on the y-axis. The slope of the correlation is roughly 4, demonstrating that using a diffuse reflectance geometry as contemplated by the present disclosure increases the signal and provides a more accurate SAF reading compared to a purely reflective geometry as known in the prior art. More specifically, the current standard for measuring skin autofluorescence measures the intensity of the fluoresced light at a detector at a distance of several centimeters from the skin, and normalizes this to the intensity of reflectance light at a point similarly distant from the skin. In contrast, the wearable device of the present disclosure for SAF measurement uses a diffuse optical design. This is an important differentiator from existing designs, as fluorescence is inherently diffuse. This is because during fluorescence the direction of the emission photon is independent of the direction of the excitation photon. By normalizing the fluorescence to the correct type of reflectance, noise caused by variation in specular reflectivity (glossiness or shininess) is eliminated, reducing error. Further measured light has necessarily traveled through the skin, increasing the fraction of light which will be absorbed and subsequently fluoresced by the skin. Supporting this explanation, a four-fold larger slope is shown for the device of the present disclosure compared to devices using specular reflectance normalization, as illustrated in FIG. 10.

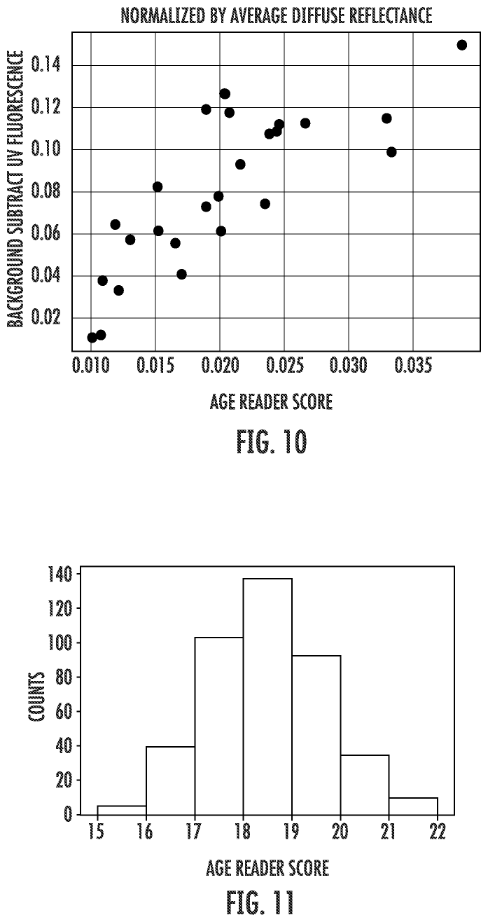

FIG. 11 is a bar graph showing the variability and lack of accuracy of advanced glycation end product (AGE) reader score with prior art devices that do not provide for continuous measurement. The current standard for SAF measurement is to conduct a single or a few measurements in a clinical setting. These measurements are inherently noisy, with a coefficient of variability of roughly 6.8%. Risk classes are separated by only 15-20%. This means that as many as 15% of users will get significantly inaccurate results. By averaging over many independent measurements, and only reporting the average result, the variability is reduced to 0.3%. This means that less than 1% of users will receive significantly inaccurate results. Additionally, continuous monitoring such as that contemplated by the devices of the present disclosure adds an important dimension to the data, creating the possibility of understanding the impact on physiological cycles including the circadian rhythm and monthly cycles.

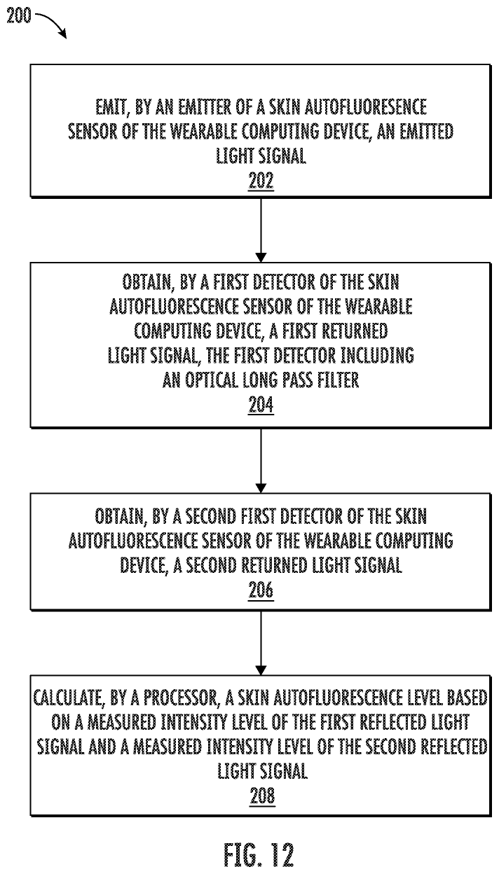

Referring now to FIG. 12, a flow diagram of a method 200 for measuring skin autofluorescence is described. The method 200 may be implemented using, for instance, the processors 150, memory 160, data 162, and/or instructions 164 discussed above with reference to FIG. 3. FIG. 12 depicts steps performed in a particular order for purposes of illustration and discussion. Those of ordinary skill in the art, using the disclosures provided herein, will understand that various steps of the method 200 may be adapted, modified, rearranged, performed simultaneously or modified in various ways without deviating from the scope of the present disclosure.

At (202), the method 200 includes emitting, by an emitter of a skin autofluorescence sensor of the wearable computing device, an emitted light signal having a wavelength ranging from about 300 nanometers to about 900 nanometers.

At (204), the method 200 includes obtaining, by a first detector of the skin autofluorescence sensor of the wearable computing device, a first returned light signal, where the first detector includes an optical long pass filter.

At (206), the method 200 includes obtaining, by a second detector of the skin autofluorescence sensor of the wearable computing device, a second returned light signal.

At (208), the method 200 includes determining, by a processor, a ratio of a measured intensity level of the first returned light signal to a measured intensity level of the second returned light signal to determine a level of autofluorescence present below a surface of a user's skin.

In summary, the existing technologies for measuring skin autofluorescence primarily measure the skin surface. This restricts the information to be only about chemistry in the epidermis, with minimal information about deeper layers of skin (e.g., dermis, subcutaneous tissue, etc.). This limitation is exacerbated by the poor penetration depth of ultraviolet light in skin. By putting the optical components closer to the skin and blocking light from reflecting directly off the skin surface into the light detector, that signal source is mostly, if not completely, eliminated in the devices contemplated by the present disclosure. This means the signal contains more information about deeper skin layers, not just the epidermis, leading to more accurate and useful SAF intensity data.

Aspects of the above-described example embodiments may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape; optical media such as CD ROM disks, Blue-Ray disks, and DVDs; magneto-optical media such as optical discs; and other hardware devices that are specially configured to store and perform program instructions, such as semiconductor memory, read-only memory (ROM), random access memory (RAM), flash memory, USB memory, and the like. Examples of program instructions include both machine code, such as produced by a compiler, and files containing higher level code that may be executed by the computer using an interpreter. The program instructions may be executed by one or more processors. The described hardware devices may be configured to act as one or more software modules in order to perform the operations of the above-described embodiments, or vice versa. In addition, a non-transitory computer-readable storage medium may be distributed among computer systems connected through a network and computer-readable codes or program instructions may be stored and executed in a decentralized manner. In addition, the non-transitory computer-readable storage media may also be embodied in at least one application specific integrated circuit (ASIC) or Field Programmable Gate Array (FPGA).

Each block of the flowchart illustrations may represent a unit, module, segment, or portion of code, which comprises one or more executable instructions for implementing the specified logical function(s). It should also be noted that in some alternative implementations, the functions noted in the blocks may occur out of order. For example, two blocks shown in succession may in fact be executed substantially concurrently (simultaneously) or the blocks may sometimes be executed in the reverse order, depending upon the functionality involved.

While the disclosure has been described with respect to various example embodiments, each example is provided by way of explanation, not limitation of the disclosure. Those skilled in the art, upon attaining an understanding of the foregoing, can readily produce alterations to, variations of, and equivalents to such embodiments. Accordingly, the disclosure does not preclude inclusion of such modifications, variations and/or additions to the disclosed subject matter as would be readily apparent to one of ordinary skill in the art. For example, features illustrated or described as part of one embodiment can be used with another embodiment to yield a still further embodiment. Thus, it is intended that the disclosure covers such alterations, variations, and equivalents.

What is claimed is:

1. A wearable computing device for measuring skin autofluorescence, the wearable computing device comprising:
a skin autofluorescence sensor comprising:
(i) one or more emitters configured to output one or more emitted light signals; and
(ii) a first detector configured to receive a first returned light signal, the first detector including an optical long pass filter; and
(iii) a second detector configured to receive a second returned light signal;
a light blocking material disposed between the one or more emitters and the first detector, the second detector, or both; and
a processor configured to calculate a skin autofluorescence level based on a measured intensity level of the first returned light signal and a measured intensity level of the second returned light signal.

2. The wearable computing device of claim 1, wherein the one or more emitted light signals has a wavelength ranging from about 300 nanometers to about 900 nanometers.

3. The wearable computing device of claim 2, wherein the wavelength ranges from about 350 nanometers to about 500 nanometers.

4. The wearable computing device of claim 1, wherein the first detector and/or the second detector are each separated from the one or more emitters by a distance ranging from about 0.5 millimeters to about 6 millimeters.

5. The wearable computing device of claim 1, wherein the skin autofluorescence level is measured continuously.

6. The wearable computing device of claim 1, wherein the skin autofluorescence sensor is in direct contact with the user's skin.

7. The wearable computing device of claim 1, wherein the one or more emitted light signals penetrates to the user's dermis.

8. The wearable computing device of claim 1, wherein the one or more emitted light signals penetrates to the user's subcutaneous tissue.

9. The wearable computing device of claim 1, wherein the one or more emitted light signals penetrates beneath the user's skin by an average distance of about 0.01 millimeters to about 3 millimeters.

10. The wearable computing device of claim 1, wherein the optical long pass filter prevents light having a wavelength that is equal to the wavelength of the one or more emitted light signals emitted by the one or more emitters from reaching the first detector.

11. The wearable computing device of claim 1, further comprising a photoplethysmography (PPG) sensor.

12. A method for measuring skin autofluorescence with a wearable computing device, the method comprising:
emitting, by one or more emitters of a skin autofluorescence sensor of the wearable computing device, one or more emitted light signals;
obtaining, by a first detector of the skin autofluorescence sensor of the wearable computing device, a first returned light signal, the first detector including an optical long pass filter;
obtaining, by a second detector of the skin autofluorescence sensor of the wearable computing device, a second returned light signal; and
calculating, by a processor, a skin autofluorescence level based on a measured intensity level of the first returned light signal and a measured intensity level of the second returned light signal.

13. The method of claim 12, wherein the one or more emitted light signals has a wavelength ranging from about 300 nanometers to about 900 nanometers.

14. The method of claim 13, wherein the wavelength ranges from about 350 nanometers to about 500 nanometers.

15. The method of claim 12, wherein the first detector and the second detector are each separated from the one or more emitters by a distance ranging from about 0.5 millimeters to about 6 millimeters.

16. The method of claim 12, wherein the skin autofluorescence level is measured continuously.

17. The method of claim 12, wherein the skin autofluorescence sensor is in direct contact with the user's skin.

18. The method of claim 12, wherein the one or more emitted light signals penetrates to the user's dermis, subcutaneous tissue, or both.

19. The method of claim 12, wherein the one or more emitted light signals penetrates beneath the user's skin by an average distance of about 0.01 millimeters to about 3 millimeters.

20. The method of claim 12, wherein the optical long pass filter prevents light having a wavelength that is equal to the wavelength of the one or more emitted light signals emitted by the one or more emitters from reaching the first detector.
